(19) United States
(12) Patent Application Publication
(10) Pub. No.: US 2022/0054680 A1
(43) Pub. Date: Feb. 24, 2022

(54) Retrofit sanitary handle

(71) Applicant: Dajcor Aluminum Ltd., Chatham (CA)

(72) Inventor: William Loucks, Chatham (CA)

(73) Assignee: Dajcor Aluminum Ltd.

(21) Appl. No.: 16/873,941

(22) Filed: Aug. 20, 2020

(51) Int. Cl. A61L 2/238; C09J 7/29; C09J 7/38; B62B 5/06

(52) U.S. Cl. CPC A61L 2/238; C09J 7/29; C09J 7/385; C09J 7/38; B62B 5/06; C09J 2483/00; C09J 2400/243; C09J 2475/006; C09J 2423/046; C09J 2411/006; C09J 2433/00; C09J 2400/163

(57) ABSTRACT

A sanitary covering is retrofit over an existing handle or railing, particularly a shopping cart handle A stability-enhancing element in the form of compression foam is adhesively secured to an existing handle. A metal cover, such as of anodized aluminum, with a pathogen-eliminating outer surface (such as a biocidal or antimicrobial coating), jackets the compression foam. The cover may be in two parts having elements interlocking them together so as to be securely attached together and securely engage the compression foam. A method of retrofitting a sanitary covering over an existing handle or rail is also provided utilizing a stability-enhancing element and a metal cover with a biocidal or antimicrobial outer coating.

### BACKGROUND AND SUMMARY OF THE INVENTION

There are many surfaces that people touch in normal life that have the possibility of transmitting disease or illness-causing pathogens. For examples microbes (bacteria, protozoa, fungi, algae, amoebas, and slime molds), viruses, and like harmful organisms/pathogens can exist on handles, rails, and other surfaces for significant periods of time and can be transferred onto a human's skin when touched. If the human then touches her/his face, or the pathogen otherwise comes into contact with sensitive tissue on the human's body, illness or disease may ensue. Common surfaces with this issue include shopping cart handles, hospital bed railings, fuel pump handles, commercial building door handles, and staircase railings.

There have been many proposals for dealing with the problem of pathogen transfer from a handle or railing to a human user. For example U.S. Pat. No. 6,817,066 provides a removable foam rubber grip for a shopping cart handle that prevents the shopper's hands from coming into contact with a conventional grocery cart handle. U.S. Pat. Nos. 5,215,319 and 5,820,142 provide removable rigid plastic sanitary coverings for a shopping cart handle, and U.S. Pat. No. 8,109,524 utilizes an elongated flexible shopping cart handle cover with an interior anti-bacterial composition. U.S. Pat. No. 10,166,158 shows a rail cover assembly having top and bottom components made of antimicrobial or biocidal materials that couple together around railings to provide a sanitary gripping surface, and US Patent Publication No. 2005/0267233 teaches using a wide variety of antimicrobial components on a handle to allow sanitary usage of the handle. While all of these proposals can be effective under some circumstances, the invention seeks to provide a system and method that are more robust and permanent than are provided by most of the above teachings and very effectively prevent pathogens from transferring from a handle or rail to a human's skin.

According to one aspect of the present invention a system for sanitizing a handle or railing comprises a retrofit covering for the handle or railing that includes a stability-enhancing interior element, and a cover of metal (preferably aluminum) that has a pathogen-eliminating outer surface. The system of the invention is particularly applicable to shopping cart handles, but is useful for a wide variety of other handles and rails.

The stability-enhancing interior element preferably comprises compression foam which operatively engages an existing handle or railing, such as by being attached thereto with an adhesive. Surrounding/jacketing the interior compression foam is a cover of a metal such as anodized aluminum with a pathogen-eliminating outer surface, such as an antimicrobial or biocidal coating including those disclosed in US Patent Publ. 2005/0267233, U.S. Provisional Application Ser. No. 62/946,272 filed Dec. 10, 2019 and entitled “Process For Making Biocidal Metallic Materials,” U.S. Pat. No. 6,929,705, or U.S. Pat. No. 8,900,716. The aluminum cover may comprise two components which have interlocking or inter-engaging elements which allow the components to be mechanically connected together and held in place to provide a new, pathogen-eliminating surface which will be engaged by the user. Alternatively the aluminum cover could comprise a single sleeve that is crimped at one or more locations to secure it over the foam interior. According to another aspect of the invention there is provided, in combination, an existing handle or railing having an outer surface; a compression foam structure adhesively secured to the existing outer surface; and a metal cover for the compression foam having an outer surface for engagement by a user, the outer surface having pathogen-eliminating properties. Desirably the metal cover is anodized aluminum with an antimicrobial or biocidal coating on the outer surface thereof, the cover comprising two components with interlocking or inter-engaging elements to hold them together, or a single component crimped at one or more locations thereof. Typically the existing handle or railing preferably comprises a shopping cart handle, although other handles and railing may be utilized.

According to yet another aspect of the invention there is provided a method for retrofitting a sturdy sanitary cover on an existing handle or railing. The method comprises a) at least partially covering the existing handle or railing with a stability-enhancing element, and then b) jacketing the stability-enhancing element with a metal cover having an outer surface with pathogen-eliminating properties. In the method a) may be practiced by adhesively securing a compression foam to the existing handle or railing, and b) may be practiced utilizing an anodized aluminum cover with an outer coating of antimicrobial or biocidal material. In the method b) may be further practiced by interlocking together or inter-engaging two components, or by crimping a sleeve. The method is particularly applicable for use on a shopping cart handle although it could also be practiced on hospital bed railings, staircase railings, commercial building door handles, or other handles or rails.

It is the primary object of the present invention to provide a robust system for sanitizing a handle or railing, and a method of retrofitting an existing handle or railing to provide an effective pathogen-eliminating user-engaging surface. This and other objects of the invention will become clear from a detailed description of the invention, and from the appended claims.

### BRIEF DESCRIPTION OF THE DRAWINGS

FIG. 1 is a cross-sectional view of a retrofit sanitary handle system covering an existing shopping cart handle, with the dimensions of the adhesive and outer coating enlarged for ease of illustration only;

FIG. 2 is a side view of the handle system of FIG. 1; and

FIG. 3 is a perspective view of a shopping cart with the retrofit sanitary handle system of FIGS. 1 & 2 operatively provided thereon.

### DETAILED DESCRIPTION OF THE DRAWINGS

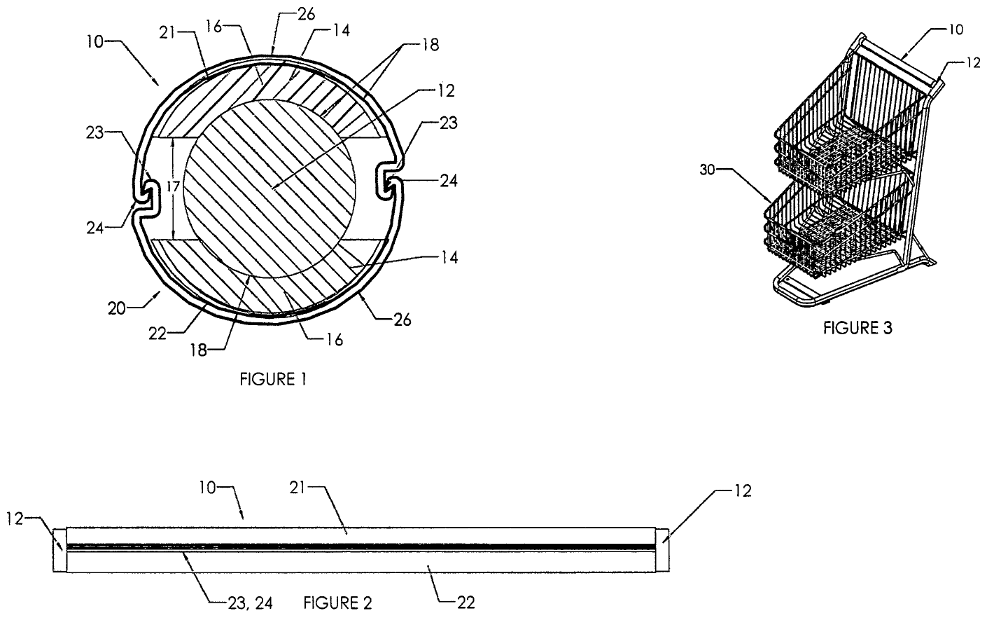

A retrofit sanitary handle system/covering according to the invention is shown generally by reference numeral 10 in FIGS. 1-3. The system 10 includes an existing handle or railing 12, which may be of metal such as aluminum, or hard plastic, and may comprise a shopping cart I handle, hospital bed railing, staircase railing, commercial building door handle, or a like handle or rail.

The system 10 includes a stability-enhancing interior element shown generally by reference numeral 14 in FIG. 1. In the preferred embodiment the stability-enhancing element comprises compression foam 16 which may be secured by adhesive 18 (shown exaggerated in size in FIG. 1 for clarity of illustration only) to the handle or railing 12. In the embodiment illustrated the foam 16 includes upper and lower portions which are spaced from each other as indicated by gaps 17 in FIG. 1. Alternatively the foam 16 could comprise a sleeve slid over the handle or railing 12, or a one-piece sheet folded over around the handle or railing 12 so that the ends thereof substantially touch.

The compression foam 16 preferably comprises a high or medium compression foam selected from the group consisting essentially of polyurethane, polyethylene, and neoprene foam, and combinations thereof, with a density of about 2-40 lbs./ft³ and which requires a pressure of about 4-15 psi to compress it 25%, preferably a density of about 8-30 lbs./ft³ and requiring a psi of about 6-12 to compress it 25%. The adhesive 18 preferably comprises an acrylic and/or silicone adhesive with an effective operating range of −40 to 120° F. The surface of the handle or railing 12 is cleaned, such as with a liquid or spray isopropanol, or an alcohol wipe, and left to dry before applying adhesive 18, and then the foam 16 to the adhesive 18.

Jacketing the compression foam 16 and securely in engagement therewith is a metal cover shown generally by reference numeral 20 in FIG. 1. The metal may be selected from aluminum (e.g. 6060-T5) such as anodized aluminum, titanium, stainless steel, or a variety of other metals, with anodized aluminum preferred. In the embodiment illustrated in FIG. 1 the cover 20 is formed by two components, an upper component 21, and a lower component 22 which, as seen in FIG. 1, can be substantially identical. Mechanical elements are preferably provided to connect the components 21, 22 by interlocking or inter-engaging them together. In the preferred embodiment illustrated in FIG. 1 the upper component 21 has one hook-shaped end 23 which receives the end projection 24 of the lower component 22, and vice-versa for the lower component 22. Alternatively one component 21, 22 can have two hook-shaped ends 23 and the other component two end projections 24.

The interlocking/inter-engaging ends 23, 24 are provided in the gaps 17 between the upper and lower portions of the foam 16, as seen in FIG. 1 so that the components 21, 22. snap together, making a “clicking” sound when they do so. The ends 23, 24 securely fit together so that they will not separate during normal use of the handle system 10, by pressing the projections 24 into operative association with the hooks 23. The fit between the projections 24 and hooks 23 may be clearance, transition, or interference, preferably a transition or interference fit. In any case preferably the cover 20 is held securely in place, and securely engages the foam 16. Optionally another layer (not shown) of the same adhesive as 18 may be provided between the foam 16 and the interiors of components 21, 22.

As an alternative to the components 21, 22, the cover 20 may be provided as a sleeve and crimped into contact with the foam substantially continuously along its length, or at one or more spaced locations.

The outer surface of the metal cover 20 has a pathogen-eliminating coating 26, shown greatly exaggerated in size in FIG. 1 for clarity of illustration only. The coating 26 may be a known biocidal or antimicrobial coating such as provided by the UmanProtek™ technology of A3 Surfaces of Chicoutimi, QC, Canada, and/or provided by a variety of known or to be developed techniques, such as shown in US Patent Publ. 2005/0267233, U.S. Provisional Application Ser. No. 62/946,272 filed Dec. 10, 2019 and entitled “Process For Making Biocidal Metallic Materials,” U.S. Pat. No. 6,929,705, and/or U.S. Pat. No. 8,900,716, the disclosures of which are hereby incorporated by reference herein.

The covering 10 according to the invention is shown in side view in FIG. 2. The length thereof will, of course, depend upon the length of the handle or rail 12 and the covering 10 may terminate just short of the ends of the handle or rail 12, as schematically illustrated in FIG. 2. As shown in FIG. 3 the covering 10 is preferably applied to the handle 12 of a conventional shopping cart 30.

According to the method of the invention, there is provided a method for retrofitting a sturdy sanitary covering 10 on an existing handle or railing 12. The method comprises: a) at least partially covering the existing handle or railing 12 with a stability-enhancing element 16 (or 16/18) as shown in FIG. 1; and then b) jacketing the stability-enhancing element 16 with a metal cover 20 having an outer surface (e. g. coating 26) with pathogen-eliminating properties.

In the method a) may be practiced by adhesively (18) securing a compression foam 16 to the existing handle or railing 12. Also in the method b) may be practiced utilizing an anodized aluminum cover 20 with an outer coating 26 of antimicrobial or biocidal material. In the method b) may be further practiced by interlocking together or inter-engaging two components 21, 22 of the cover 20 (such as by using hook 23 and projection 24 ends thereof in a transition or interference fit, and snap together with a “clicking” sound) so that they are securely held together and preferably in secure contact with the stability-enhancing element 16; or alternatively b) is further practiced by tightly crimping a sleeve of aluminum into contact with the stability-enhancing element. Most desirably, a) and b) are practiced on a shopping cart handle 12 as the existing handle or railing, as seen in FIG. 3 for shopping cart 30.

While the invention has been herein shown and described in what is presently conceived to be preferred embodiments thereof it is to be understood that the system, combination, and method of the invention are to be accorded the broadest interpretation of the appended claims so as to encompass all equivalent structures and procedures limited only by the prior art. This disclosure is also to be interpreted so as to encompass all narrower ranges within a broad range; for example a foam density of about 2-40 lbs./ft.³ specifically covers 3.0-33.2, 8.4-30.5, 1.95-6.8, and all other specific ranges within the broad range.

What is claimed is:

1. A system for sanitizing a handle or railing comprising a retrofit covering for the handle or railing; said retrofit covering including a stability-enhancing interior element, and a cover of metal that has a pathogen-eliminating outer surface.

2. The system of claim 1 wherein the handle or railing comprises a shopping cart handle.

3. The system of claim 1 wherein said stability-enhancing interior element comprises compression foam which operatively engages the existing handle or railing.

4. The system of claim 3 wherein said compression foam is attached to the handle or railing with an adhesive.

5. The system of claim 3 wherein said cover of metal comprises an anodized aluminum cover with an antimicrobial or biocidal coating on the outer surface thereof.

6. The system of claim 5 wherein said aluminum cover comprises first and second components, and mechanically interlocking or inter-engaging elements which hold said first and second components securely together and engaging said compression foam.

7. The system of claim 4 wherein said compression foam comprises a high or medium compression foam selected from the group consisting essentially of polyurethane, polyethylene, and neoprene foam, and combinations thereof, with a density of about 2-40 lbs./ft.³ and which requires a pressure of about 4-15 psi to compress it 25%; and wherein said adhesive comprises an acrylic and/or silicone adhesive.

8. A combination:
an existing handle or railing having an outer surface;
a compression foam component adhesively secured to said existing handle or railing outer surface; and
a metal cover for said compression foam, said cover having an outer surface for engagement by a user, said outer surface having pathogen-eliminating properties.

9. A combination as recited in claim 8 wherein said existing handle or railing comprises a shopping cart handle.

10. A combination as recited in claim 8 wherein said metal cover comprises anodized aluminum with an antimicrobial or biocidal coating on the outer surface thereof.

11. A combination as recited in claim 10 wherein said cover comprises two components with interlocking or inter-engaging elements to hold them together.

12. A combination as recited in claim 10 wherein said cover comprises a single component crimped at one or more locations thereof to securely hold it in contact with said compression foam.

13. A combination as recited in claim 8 wherein said compression foam comprises a high or medium compression foam selected from the group consisting essentially of polyurethane, polyethylene, and neoprene foam, and combinations thereof, with a density of about 2-40 lbs./ft.³ and which requires a pressure of about 4-15 psi to compress it 25%; and wherein said adhesive comprises an acrylic and/or silicone adhesive.

14. A method for retrofitting a sturdy sanitary cover on an existing handle or railing comprising:
a) at least partially covering the existing handle or railing with a stability-enhancing element; and then
b) jacketing the stability-enhancing element with a metal cover having an outer surface with pathogen-eliminating properties.

15. A method as recited in claim 14 wherein a) is practiced by adhesively securing a compression foam to the existing handle or railing.

16. A method as recited in claim 15 wherein b) is practiced utilizing an anodized aluminum cover with an outer coating of antimicrobial or biocidal material.

17. A method as recited in claim 14 wherein b) is practiced utilizing an anodized aluminum cover with an outer coating of antimicrobial or biocidal material.

18. A method as recited in claim 17 wherein b) is further practiced by interlocking together or inter-engaging two components of the cover so that they are in secure contact with each other and the stability-enhancing element.

19. A method as recited in claim 17 wherein b) is further practiced by tightly crimping a sleeve of aluminum into contact with the stability-enhancing element.

20. A method as recited in claim 14 wherein a) and b) are practiced on a shopping cart handle as the existing handle or railing.
